(19) United States
(12) Patent Application Publication
(10) Pub. No.: US 2005/0169991 A1
(43) Pub. Date: Aug. 4, 2005

(54) Sustained release torsemide dosage forms

(75) Inventors: Pradeep Sanghvi, Schererville, IN (US); Sara Ketsela, Danbury, CT (US); Thomas Sciascia, Belmont, MA (US); Theodore Joseph Jaworski, Cary, NC (US)

Correspondence Address: DAVIDSON, DAVIDSON & KAPPEL, LLC, 485 SEVENTH AVENUE, 14TH FLOOR, NEW YORK, NY, 10018, US

(73) Assignee: Penwest Pharmaceuticals Co., Patterson, NY (US)

(21) Appl. No.: 11/009,800

(22) Filed: Dec. 10, 2004

Related U.S. Application Data
(60) Provisional application No. 60/529,138, filed on Dec. 12, 2003.

(51) Int. Cl. A61K 9/22; A61K 31/736

(52) U.S. Cl. 424/468; 514/54

(57) ABSTRACT

A sustained release pharmaceutical formulation comprising torsemide or pharmaceutically acceptable salt thereof.

This application claims priority from U.S. Provisional Application No. 60/529,138, filed Dec. 12, 2003, the disclosure of which is hereby incorporated by reference in its entirety.

### BACKGROUND OF THE INVENTION

Congestive heart failure affects 1.7% of the U.S. population, 4.6 million people have chronic heart failure, there are 550,000 new cases per annum and approximately 60% are over 70 years of age. The etiological causative factors are coronary heart disease, hypertension, cardiac valvular disease, arrhythmias, cardiomyopathy and diabetes. It is associated with high mortality rate. In the U.S. the median survival following onset of CHF is 1.7 years in men and 3.2 years in women. Data generated from Scotland shows a 3-year mortality rate after first hospitalization for CHF patients age 65 years and older is approximately 66%.

Fluid overload resulting in pulmonary and/or peripheral edema is a primary cause of hospitalization among patients with chronic heart failure. Thus diuretics play an essential role in this multi therapeutic treatment of this disease.

Loop diuretics typically are the drugs of choice. Examples of these drugs commonly prescribed with their half-lives are: Bumetanide—short acting 1½ hours, Furosemide—4½ hours and Torsemide—6 hours. Of these three, Furosemide is the most commonly prescribed in the treatment of congestive cardiac failure.

As described in Michael D. Murray, et al., “Open-label Randomized Trial of Torsemide Compared with Furosemide Therapy for Patients with Heart Failure”, The American Journal of Medicine, Volume 111, pp. 513-520 (November 2001), the disclosure of which is hereby incorporated by reference in its entirety, furosemide has erratic oral absorption, with bioavailability of 11% to 90%, and studies since the 1970s have documented substantial variability in furosemide absorption not only between, but also within patients, that is accompanied by variability in the natriuretic response. Alternatively, Murray, et al. describe torsemide as having a more complete and much less variable bioavailability (76% to 96%).

Torsemide is a loop diuretic approved for edema associated with congestive heart failure, renal disease (e.g., chronic renal failure), hepatic disease, and hypertension. Treatment of congestive heart failure is the most significant and widely used indication for torsemide. For congestive heart failure, the recommended dose of torsemide is 10 mg to 20 mg once daily titrated upwards by doubling the dose.

Common problems with diuretics are acute and chronic tolerance. Acute tolerance occurs in a breaking phenomena associated with a shift to the right of the dose response curve and occurs after initial dosing. Chronic tolerance occurs after 5-10 weeks of dosing and is associated with tubular hypertrophy and sodium rebound phenomena. Although multiple physiological mechanisms are involved in this phenomena, acute volume depletion is the main stimulus to this phenomena.

U.S. patent publication No. 2003/0152622 A1 describes formulations of an erodible gastric retentive oral diuretic, and exemplifies furosemide as the diuretic.

In view of the above, there exists a need in the art for improving the effectiveness of diuretic therapy.

### OBJECTS AND SUMMARY OF THE INVENTION

It is an object of the present invention to provide a sustained release oral dosage form for torsemide or a pharmaceutically acceptable salt thereof.

It is a further object of certain embodiments of the present invention to provide a method for preparing a bioavailable sustained release oral dosage form for torsemide or a pharmaceutically acceptable salt thereof.

It is a further object of certain embodiments of the present invention to provide a method of treatment of edema via administration of torsemide or a pharmaceutically acceptable salt thereof in a sustained release oral dosage form to a human patient in need of such treatment.

It is a further object of certain embodiments of the present invention to provide a sustained release oral diuretic dosage form which does not have an unfavorable pharmacokinetic profile such as an erratic oral absorption and varying bioavailability.

It is a further object of certain embodiments of the present invention to provide a method of treatment of congestive heart failure (CHF) via administration of torsemide or a pharmaceutically acceptable salt thereof in a sustained release oral dosage form to a human patient in need of such treatment.

It is a further object of certain embodiments of the present invention to provide a sustained release oral dosage form which is suitable for providing, when combined with torsemide or a pharmaceutically acceptable salt thereof, a sustained release formulation which provides therapeutically effective blood levels of the torsemide for treating edema and/or congestive heart failure for, e.g., about 12 to about 24 hours.

The above-mentioned objects and others are achieved by virtue of the present invention, which is directed in part to a sustained release oral dosage form comprising a therapeutically effective amount of a torsemide or a pharmaceutically acceptable salt thereof and a sustained release excipient which provides for the release of the torsemide or pharmaceutically acceptable salt thereof for about 12 to about 24 hours when the dosage form is exposed to an environmental fluid.

In certain embodiments, the sustained release oral dosage form of the present invention provide an in-vitro dissolution rate when measured by USP 26 (2003) dissolution Apparatus type III, in pH change media with an agitation of 15 dpm in 250 ml and at 37° C. which is from 0 to about 50% torsemide released after 1 hour; from about 1 to about 60% torsemide released after 3 hours; from about 5 to about 70% torsemide released after 7 hours; from about 10 to about 95% torsemide released after 12 hours; not less than about 25% torsemide released after 16 hours; and not less than about 35% torsemide released after 24 hours.

In certain embodiments, the sustained release oral dosage form of the present invention provides a mean urinary excretion rate of torsemide of at least about 200 μg/hr for about 4 to about 20 hours, preferably for about 8 to about 18 hours, more preferably for about 12 to about 16 hours after single dose oral administration of the sustained release oral dosage form to human subjects.

In certain preferred embodiments, the sustained release oral dosage form of the present invention provides a mean urinary excretion rate of torsemide of at least about 700 μg/hr for about 4 to about 12 hours, preferably for about 8 to about 12 hours after single dose oral administration of the sustained release oral dosage form to human subjects.

In certain embodiments the sustained release oral dosage form of the present invention provides a mean urinary excretion rate of torsemide of about 210 μg/hr to about 848 μg/hr at from 0 to about 4 hours; about 290 μg/hr to about 1160 μg/hr at from about 4 to about 8 hours; about 161 μg/hr to about 778 μg/hr at from about 8 to about 12 hours; about 122 μg/hr to about 301 μg/hr at from about 12 to about 16 hours; about 133 82 g/hr to about 323 μg/hr at from about 16 to about 20 hours; and about 64 μg/hr to about 182 μg/hr at from about 20 to about 24 hours after single dose oral administration of the sustained release oral dosage form to human subjects.

In certain preferred embodiments, the sustained release oral dosage form of the invention provides a mean sodium (Na⁺) excretion rate of from about 48 mmol/hr to about 81 mmol/hr, preferably from about 60 mmol/hr to about 70 mmol/hr at from 0 to 4 hours, and from about 2 mmol/hr to about 13 mmol/hr, preferably from about 4 mmol/hr to about 8 mmol/hr at from 12 to 16 hours after single dose oral administration of the sustained release oral dosage form to human subjects.

In certain embodiments the sustained release oral dosage form of the present invention provides a mean Cmax of torsemide of from about 1 μg/ml to about 7 μg/ml, preferably form about 1.6 μg/ml to about 6.2 μg/ml, more preferably from about 3.9 μg/ml to about 4.7 μg/ml per 100 mg of torsemide upon single dose oral administration to human subjects.

In certain embodiments the sustained release oral dosage form of the present invention provides a mean Tₘₐₓ of torsemide at from about 1 to about 8 hours, preferably from about 1.7 to about 5.7 hours, more preferably at from about 1.7 to about 5.2 hours after single dose oral administration to human subjects.

In certain embodiments the sustained release oral dosage form of the present invention provides a mean AUC₍₀₋₂₄₎ of from about 10 μg.h/ml to about 40 μg.h/ml, preferably from about 13.9 μg.h/ml to about 34.1 μg.h/ml, more preferably from about 22.5 μg.h/ml to about 34.1 μg.h/ml per 100 mg torsemide upon single dose oral administration to human subjects.

In certain embodiments, the present invention is further directed to a method of treating a human patient for edema by orally administering a sustained release oral dosage form as set forth herein to a patient in need of such treatment.

In certain embodiments, the present invention is further directed to a method of treating a human patient for congestive heart failure by orally administering a sustained release oral dosage form as set forth herein to a patient in need of such treatment.

In certain embodiments, the present invention is further directed to a method of treating a human patient for hypertension by orally administering a sustained release oral dosage form as set forth herein to a patient in need of such treatment.

In certain embodiments, the present invention is further directed to a method of preventing or decreasing sodium rebound phenomena typically associated with the administration of loop diuretics comprising orally administering a sustained release oral dosage form as set forth herein to a patient in need of diuretic treatment.

In certain preferred embodiments, the sustained release oral dosage form is administered in the fed state. In alternative preferred embodiments, the sustained release oral dosage form is administered in the fasted state.

In certain embodiments, the methods of the invention further include administering the dosage form to the human patient in the morning, preferably providing for therapeutically effective blood levels of torsemide throughout the day causing excretion during the hours that the patient is awake.

In certain embodiments, the sustained release excipient is incorporated into a matrix with torsemide or a pharmaceutically acceptable salt thereof which matrix provides for the sustained release of the torsemide or a pharmaceutically acceptable salt thereof when exposed to an environmental fluid.

In certain embodiments, the sustained release excipient is a sustained release coating which is coated over e.g., a substrate comprising torsemide or pharmaceutically acceptable salt thereof, wherein the sustained release coating provides for the sustained release of the torsemide or pharmaceutically acceptable salt thereof when exposed to an environmental fluid.

In certain embodiments, the sustained release oral dosage form includes both a matrix and a coating which provide for the sustained release of the torsemide or pharmaceutically acceptable salt thereof when exposed to an environmental fluid.

In certain embodiments, the sustained release oral dosage form of the invention further comprises an immediate release component of torsemide or pharmaceutically acceptable salt thereof in addition to the sustained release form of torsemide or pharmaceutically acceptable salt thereof. In certain preferred embodiments, the sustained release oral dosage form is a bilayer tablet, wherein both layers include the torsemide or pharmaceutically acceptable salt thereof and wherein one layer provides for the immediate release of the torsemide or pharmaceutically acceptable salt thereof and the other layer provides for the sustained release of the torsemide or pharmaceutically acceptable salt thereof upon exposure to an environmental fluid.

In certain preferred embodiments, from about 10% to about 40%, preferably from about 20% to about 30% of the total amount torsemide or pharmaceutically acceptable of the sustained release oral dosage form is included in the immediate release component.

In certain preferred embodiments, the sustained release excipient comprises a gelling agent comprising at least one natural or synthetic gum, the dosage form providing a sustained release of the torsemide or a pharmaceutically acceptable salt thereof when exposed to an environmental fluid. In certain preferred embodiments, the gelling agent comprises a heteropolysaccharide gum, a homopolysaccharide gum, or a combination thereof. Preferably in combination, the homopolysaccharide gum is capable of cross-linking said heteropolysaccharide gum when exposed to an environmental fluid.

In certain preferred embodiments, the sustained release excipient further comprises an inert diluent selected from, e.g., a monosaccharide, a disaccharide, a polyhydric alcohol, or mixtures thereof.

In certain preferred embodiments, the sustained release formulation of the present invention further comprises an ionizable gel strength enhancing agent. Preferably the ionizable gel strength enhancing agent is included in the sustained release excipient.

In a preferred embodiment of the present invention the ratio of torsemide or a pharmaceutically acceptable salt thereof to gelling agent is from about 5:1 to about 1:10, preferably about 3:1 to about 1:6 or from about 1:0.5 to about 1:2, more preferably about 1:1.

In a preferred embodiment of the present invention, the ratio of inert diluent to gelling agent is from about 1:8 to about 8:1, preferably from about 1:3 to about 3:1. In certain preferred embodiments, the present invention is further directed to a method for preparing the sustained release torsemide or pharmaceutically acceptable salt thereof formulations described herein.

In certain preferred embodiments, the present invention is further directed to a method for providing a sustained release formulation of a torsemide or a pharmaceutically acceptable salt thereof comprising preparing a matrix comprising a gelling agent, optionally an ionizable gel strength enhancing agent, and an inert pharmaceutical diluent; and thereafter adding torsemide or a pharmaceutically acceptable salt thereof, optionally a pharmaceutically acceptable surfactant, optionally a wetting agent, and optionally a pH modifying agent. Thereafter the resulting mixture is tableted, such that a gel matrix is created when the tablet is exposed to an environmental fluid and such that the tablets each contain a therapeutically effective amount of the medicament. In certain embodiments, an immediate release component is included in the tablet formulation. Preferably, a first portion of the medicament (e.g., torsemide) is introduced during the granulation of the excipient, and a second portion of the medicament (e.g., torsemide) is introduced extragranularly, or after the granulation step. Such an embodiment preferably provides an initial rapid release of the medicament. Most preferably, the immediate release component is included during tableting, forming a bilayer tablet (e.g., having a sustained release layer and an immediate release layer). The resulting tablet provides therapeutically effective blood levels of the medicament for at least about 12 hours, and preferably about 24 hours, more preferably from about 12 to about 16 hours after oral adminstration.

In certain embodiments, the present invention further comprises the sustained release excipient being granulated with an ionizable gel strength enhancing agent and/or a solution or a dispersion of a hydrophobic material in an amount effective to slow the hydration of the gelling agent without disrupting the hydrophilic matrix.

By “sustained release” it is meant for purposes of the present invention that the torsemide or a pharmaceutically acceptable salt thereof is released from the formulation at a controlled rate such that therapeutically beneficial blood levels (at least minimally effective levels and below toxic levels) of the torsemide are maintained over an extended period of time, e.g., for about a 12 hour to about 24 hours, such that the formulations are suitable for once a day administration.

By “bioavailable” it is meant for purposes of the present invention that the therapeutically active medicament is absorbed from the sustained release formulation and becomes available in the body at the intended site of drug action.

The term “environmental fluid” is meant for purposes of the present invention to encompass a fluid of an environment of use, e.g., an aqueous solution, or gastrointestinal fluid.

The term “pH modifying agent” is meant for purposes of the present invention to mean any substance which decreases the ionization of the medicament, whereby the release of the drug from the hydrogel matrix and into solution is facilitated.

The term “Cmax” is meant for purposes of the present invention to mean the maximum plasma concentration of a medicament achieved after single dose administration of a dosage form in accordance with the present invention.

The term “Tmax” is meant for purposes of the present invention to mean the elapsed time from administration of a dosage form to the time the Cmax of the medicament is achieved.

The term “human subject” is meant for purposes of the present invention to be a healthy volunteer, such as an individual who is not known to suffer any illness relevant to the medication being administered in a study being performed and who is able to understand and give valid consent to the study.

The term “human patient” is meant for purposes of the present invention to be an individual who suffers from an illness relevant to the medication being administered.

The term “pH change media” is meant for purposes of the present invention to be a dissolution media which, when used in accordance with USP type III dissolution apparatus described herein, has a pH of 1.5 at the outset of the dissolution test and is changed from 1.5 to 4.5 after 1 hour and from 4.5 to 7.5 after 3 hours.

### DETAILED DESCRIPTION

The sustained release oral dosage forms of the present invention preferably provide for therapeutic levels of torsemide, which are suitable for the treatment of edema, preferably edema associated with conditions such as congestive heart failure, liver disease, and/or renal disease.

In certain embodiments, the sustained release oral dosage form of the invention provides therapeutically effective levels of torsemide over a period at least about 12 hours, and for about 24 hours. Preferably the sustained release oral dosage form of the invention provides therapeutically effective levels of torsemide over a period of from about 8 to about 24 hours, from about 8 to about 20 hours, preferably from about 10 to about 18 hours, more preferably from about 12 to about 16 hours, most preferably about 14 to 16 hours after single dose oral administration to human patients.

In certain preferred embodiments, the sustained release oral dosage form of the present invention provides an effective plasma level of torsemide maintained over an extended period throughout the day to maintain an effective concentration within the nephron of the kidney, promoting fluid and sodium loss over this period of time (e.g., during the time when food is ingested during the day). Preferably, this shortens the window of opportunity for the nephrons to absorb sodium over a time period during sleep when there is no food intake and hence lessens the sodium rebound phenomena.

Preferably the sustained release oral dosage form of the present invention provides a mean Cmax of torsemide of from about 1 μg/ml to about 5 μg/ml, preferably from about 1.6 μg/ml to about 4.0 μg/ml per 100 mg of torsemide upon single dose oral administration to human subjects under fasted conditions.

In certain further embodiments the sustained release oral dosage form of the present invention provides a mean Cmax of torsemide of from about 3 μg/ml to about 7 μg/ml, preferably from about 4.8 μg/ml to about 5.7 μg/ml per 100 mg of torsemide upon single dose oral administration to human subjects under fed conditions.

In certain embodiments the sustained release oral dosage form of the present invention provides a mean Tmax of torsemide at from about 1 to about 8 hours, preferably at from about 1.7 to about 5.2 hours after single dose oral administration to human subjects under fasted conditions.

In certain further embodiments the sustained release oral dosage form of the present invention provides a mean Tmax of torsemide at from 3 to about 8 hours, preferably at from about 4.8 to about 5.7 hours after single dose oral administration to human subjects under fed conditions.

In certain embodiments the sustained release oral dosage form of the present invention provides a mean AUC₍₀₋₂₄₎ of torsemide of from about 10 μg.h/ml to about 30 μg.h/ml, preferably from about 13.9 μg.h/ml to about 22.6 μg.h/ml per 100 mg torsemide upon single dose oral administration to human subjects under fasted conditions.

In certain further embodiments the sustained release oral dosage form of the present invention provides mean AUC₍₀₋₂₄₎ of torsemide of from about 25 μg.h/ml to about 40 μg.h/ml, preferably from about 31.6 μg.h/ml to about 34.1 μg.h/ml per 100 mg torsemide upon single dose oral administration to human subjects under fed conditions.

In certain embodiments the sustained release oral dosage form of the present invention provides a mean urinary excretion rate of torsemide of about 210 μg/hr to about 730 μg/hr at from 0 to about 4 hours; about 857 μg/hr to about 1160 μg/hr at from about 4 to about 8 hours; about 424 μg/hr to about 777 μg/hr at from about 8 to about 12 hours; about 122 μg/hr to about 301 μg/hr at from about 12 to about 16 hours; about 133 μg/hr to about 323 μg/hr from at about 16 to about 20 hours; and about 64 μg/hr to about 176 μg/hr at from about 20 to about 24 hours after single dose oral administration of the sustained release oral dosage form to human subjects in the fed state.

In certain embodiments the sustained release oral dosage form of the present invention provides a mean urinary excretion rate of torsemide of about 263 μg/hr to about 848 μg/hr at from 0 to about 4 hours; about 290 μg/hr to about 686 μg/hr from at from about 4 to about 8 hours; about 161 μg/hr to about 290 μg/hr at from about 8 to about 12 hours; about 155 μg/hr to about 206 μg/hr at from about 12 to about 16 hours; about 206 μg/hr to about 321 μg/hr at from about 16 to about 20 hours; and about 117 μg/hr to about 182 μg/hr at from about 20 to about 24 hours after single dose oral administration of the sustained release oral dosage form to human subjects in the fasted state.

In certain embodiments, the sustained release dosage forms of the present invention provide an in-vitro dissolution rate when measured by USP 26 (2003) dissolution Apparatus type III, in pH change media with an agitation of 15 dpm in 250 ml and at 37° C. which is from about 5 to about 44% torsemide released after 1 hour; from about 6 to about 46% torsemide released after 3 hours; from about 11 to about 54% torsemide released after 7 hours; from about 21 to about 91% torsemide released after 12 hours; not less than about 35% torsemide released after 16 hours; and not less than about 42% torsemide released after 24 hours.

In certain embodiments, the sustained release dosage forms of the present invention provide an in-vitro dissolution rate when measured by USP 26 (2003) dissolution Apparatus type III, in pH change media with an agitation of 15 dpm in 250 ml and at 37° C. which is from about 5 to about 44% torsemide released after 1 hour; from about 6 to about 46% torsemide released after 3 hours; from about 11 to about 54% torsemide released after 7 hours; from about 41 to about 91% torsemide released after 12 hours; not less than about 64% torsemide released after 16 hours; and not less than about 90% torsemide released after 24 hours.

In certain embodiments, the sustained release dosage forms of the present invention provide an in-vitro dissolution rate when measured by USP 26 (2003) dissolution Apparatus type III, in pH change media with an agitation of 15 dpm in 250 ml and at 37° C. which is from about 5 to about 32% torsemide released after 1 hour; from about 12 to about 34% torsemide released after 3 hours; from about 37 to about 54% torsemide released after 7 hours; from about 78 to about 84% torsemide released after 12 hours; not less than about 64% torsemide released after 16 hours; and not less than about 90% torsemide released after 24 hours.

Preferably the sustained release oral dosage form of the present invention provides a mean Cmax of torsemide of 1.662±1.00 μg/ml, 3.948±0.8 μg/ml, or 3.364±3.42 μg/ml per 100 mg of torsemide upon single dose oral administration to human subjects under fasted conditions.

In certain further embodiments the sustained release oral dosage form of the present invention provides a mean Cmax of torsemide of 4.800±1.93 μg/ml, 4.698±2.11 μg/ml, or 6.11±4.52 μg/ml upon single dose oral administration to human subjects under fed conditions.

In certain embodiments the sustained release oral dosage form of the present invention provides a mean Tmax of torsemide at from 5.13±5.51 hours, 1.72±1.81 hours, or 4.57±1.4 hours after single dose oral administration to human subjects under fasted conditions.

In certain further embodiments the sustained release oral dosage form of the present invention provides a mean Tmax of torsemide at from 5.67±3.44 hours, 5.19±2.69 hours, or 4.83±1.83 hours, after single dose oral administration to human subjects under fed conditions.

In certain embodiments the sustained release oral dosage form of the present invention provides a mean AUC₍₀₋₂₄₎ of torsemide of from about 13.976±3.24 μg.h/ml, 22.563±7.52 μg.h/ml, or 21.506±12.17 μg.h/ml per 100 mg torsemide upon single dose oral administration to human subjects under fasted conditions.

In certain embodiments the sustained release oral dosage form of the present invention provides a mean AUC₍₀₋₂₄₎ of torsemide of from about 31.651±15.15 μg.h/ml, 34.075±14.76 μg.h/ml, or 33.471±24.95 μg.h/ml per 100 mg torsemide upon single dose oral administration to human subjects under fed conditions.

In certain embodiments, the invention is further directed to a method of treating edema in a human patient comprising administering to said human patient a sustained release oral dosage form of comprising torsemide or a pharmaceutically acceptable salt thereof and a sustained release, such that the oral dosage form provides an mean AUC(0-24) which does not vary by more than about 50%, preferably not more that about 25%, more preferably not more than about 15% when administered to human subjects.

In certain embodiments, the invention is further directed to a method of treating edema in a human patient comprising administering to said human patient a sustained release oral dosage form of comprising torsemide or a pharmaceutically acceptable salt thereof and a sustained release excipient, such that the oral dosage form provides a mean Cmax with a variability of 0 to about 60%, from about 10 to about 60%, preferably a variability of not more than about 40%, more preferably not more than about 20% when administered to human subjects.

The sustained release oral dosage form of the present invention includes a sustained release excipient which comprises a sustained release material which provides for the sustained release of the torsemide or pharmaceutically acceptable salt thereof.

A non-limiting list of suitable sustained-release materials which may be included in a sustained-release excipient according to the invention include hydrophilic and/or hydrophobic materials, such as gums, cellulose ethers, acrylic resins, protein derived materials, waxes, shellac, sustained release polymers, and oils such as hydrogenated castor oil and hydrogenated vegetable oil. Certain sustained-release polymers include alkylcelluloses such as ethylcellulose, acrylic and methacrylic acid polymers and copolymers (such as Eudragit® by Rohm Pharma; and cellulose ethers, especially hydroxyalkylcelluloses (especially hydroxypropylmethylcellulose) and carboxyalkylcelluloses. Examples of acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Waxes include for example natural and synthetic waxes, fatty acids, fatty alcohols, and mixtures of the same (e.g., beeswax, camauba wax, stearic acid and stearyl alcohol). Examples of gums include, for example and without limitation, heteropolysaccharides such as xanthan gum(s), homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g. hydrocolloids), mixtures of any of the foregoing, and the like. Certain embodiments utilize mixtures of any of the foregoing sustained release materials in the sustained release excipient. However, any pharmaceutically acceptable hydrophobic or hydrophilic sustained-release material which is capable of imparting sustained-release of the active agent may be used in accordance with the present invention.

The sustained release oral dosage forms of the present invention can be manufactured as a suitable tablet or multiparticulate formulation utilizing procedures known to those skilled in the art which can be modified such that the dosage form provides for the release of the torsemide or pharmaceutically acceptable salt thereof over about 12 to about 24 hours when exposed to an environmental fluid. In either case, the sustained release dosage form includes a sustained release excipient which is incorporated into a matrix along with the drug (e.g., torsemide), or which is applied as a controlled release coating.

An oral dosage form according to the invention may be provided as, for example, granules, spheroids, beads, pellets (hereinafter collectively referred to as multiparticulates) and/or particles. An amount of the multiparticulates which is effective to provide the desired dose of torsemide over time may be placed in a capsule or may be incorporated in any other suitable oral solid form. In one preferred embodiment of the present invention, the controlled release dosage form comprises such particles containing or comprising the active ingredient, wherein the particles have diameter from about 0.1 mm to about 2.5 mm.

Examples of suitable multiparticulate formulations are those in which the particles comprise inert beads which are coated with the drug. Thereafter, a coating comprising the sustained release excipient is applied onto the beads. Alternatively, a spheronizing agent, together with the drug can be spheronized to form spheroids. In such embodiments, in addition to drug and spheronizing agent, the spheroids may also contain a binder. Additionally (or alternatively) the spheroids may contain a water insoluble polymer, especially an acrylic polymer, an acrylic copolymer, such as a methacrylic acid-ethyl acrylate copolymer, or ethyl cellulose.

In certain embodiments, the particles comprise normal release matrixes containing the drug. These particles are then coated with the sustained release excipient (e.g., sustained release coating).

In certain embodiments, coatings are provided to permit either pH-dependent or pH-independent release, e.g., when exposed to gastrointestinal fluid. Coatings which are pH-dependent may be used in accordance with the present invention include shellac, cellulose acetate phthalate (CAP), polyvinyl acetate phthalate (PVAP), hydroxypropylmethylcellulose phthalate, and methacrylic acid ester copolymers, and the like.

In certain preferred embodiments, the tablet core or multiparticulates containing the drug are coated with a hydrophobic material selected from (i) an alkylcellulose; (ii) an acrylic polymer; or (iii) mixtures thereof. The coating may be applied in the form of an organic or aqueous solution or dispersion. The coating may be applied to obtain a weight gain from about 2 to about 25% of the substrate in order to obtain a desired sustained release profile.

The sustained release coatings of the present invention may also include an exit means comprising at least one passageway, orifice, or the like. In certain embodiments, wherein a passageway is included in the coating, an osmotic agent may be further included in the core of the formulation. In certain embodiments, wherein the oral dosage form of the present invention comprises a passageway, preferably the dosage form is an osmotic dosage form having a push or displacement composition as one of the layers of a bilayer core for pushing the torsemide or a pharmaceutically acceptable salt thereof from the dosage form, and a semipermeable wall comprising the sustained release excipient and surrounding the core, wherein the wall has the at least one exit means or passageway for delivering the torsemide or pharmaceutically acceptable salt thereof from the dosage form. In certain embodiments, the core of the osmotic dosage form may comprise a single layer core optionally including the torsemide or a pharmaceutically acceptable salt thereof and optionally a sustained release material. In such osmotic embodiments, the torsemide or pharmaceutically may be released only through the passageway, or may be released through the passageway and the coating (e.g., through erosion of the coating and/or pore formers in the coating).

In other embodiments of the present invention, the desired controlled release of the formulation is achieved via a matrix. In certain embodiments, the matrix may be a sustained release matrix, a normal release matrix having a sustained release coating, or a combination of a sustained release matrix and a sustained release coating. The present invention may also utilize a sustained release matrix that affords in-vitro dissolution rates of the drug in a pH-dependent or pH-independent manner. The sustained release material which may be included in a matrix in addition to the drug includes those materials described above. Any pharmaceutically acceptable hydrophobic material or hydrophilic material which is capable of imparting controlled release of the active agent may be used in accordance with the matrix of the present invention.

In addition to the above ingredients, a controlled release matrix may also contain suitable quantities of other materials, e.g. diluents, lubricants, binders, granulating aids, colorants, flavorants and glidants that are conventional in the pharmaceutical art. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

In certain preferred embodiments, the sustained release excipient of the present invention comprises a gelling agent of a heteropolysaccharide such as e.g., xanthan gum, a homopolysaccharide such as e.g., locust bean gum, or a mixture of one or more hetero- and one or more homopolysaccharide(s). Heterodisperse excipients, previously disclosed in our U.S. Pat. Nos. 4,994,276, 5,128,143, and 5,135,757, may be utilized in the sustained release excipient of the present invention. For example, the sustained release excipient comprises a gelling agent of both hetero- and homo-polysaccharides which exhibit synergism, e.g., the combination of two or more polysaccharide gums producing a higher viscosity and faster hydration than that which would be expected by either of the gums alone, the resultant gel being faster-forming and more rigid.

The term “heteropolysaccharide” as used in the present invention is defined as a water-soluble polysaccharide containing two or more kinds of sugar units, the heteropolysaccharide having a branched or helical configuration, and having excellent water-wicking properties and immense thickening properties.

An especially preferred heteropolysaccharide is xanthan gum, which is a high molecular weight (>10⁶) heteropolysaccharide. Other preferred heteropolysaccharides include derivatives of xanthan gum, such as deacylated xanthan gum, the carboxymethyl ether, and the propylene glycol ester.

The homopolysaccharide gums used in the present invention which are capable of cross-linking with the heteropolysaccharide include the galactomannans, i.e., polysaccharides which are composed solely of mannose and galactose. Galactomannans which have higher proportions of unsubstituted mannose regions have been found to achieve more interaction with the heteropolysaccharide. Locust bean gum, which has a higher ratio of mannose to the galactose, is especially preferred as compared to other galactomannans such as guar and hydroxypropyl guar.

The combination of xanthan gum with locust bean gum is an especially preferred gum combination for use in the sustained release excipient of the present invention.

In certain preferred embodiments, the ratio heteropolysaccharide gum to homopolysaccharide gum is from about 1:3 to about 3:1. Preferably, the controlled release properties of the sustained release formulations of the present invention may be optimized when the ratio of heteropolysaccharide gum to homopolysaccharide material is about 1:1 or about 1:1.5, although heteropolysaccharide gum in an amount of from about 10 to about 90 percent or more by weight of the heterodisperse polysaccharide material provides an acceptable slow release product. The combination of any homopolysaccharide gums known to produce a synergistic effect when exposed to aqueous solutions may be used in accordance with the present invention. It is also possible that the type of synergism which is present with regard to the gum combination of the present invention could also occur between two homogeneous or two heteropolysaccharides.

Other acceptable gelling agents which may be used in the present invention include those gelling agents well-known in the art. Examples include vegetable gums such as alginates, gum karaya, pectin, agar, tragacanth, accacia, carrageenan, tragacanth, chitosan, agar, alginic acid, other polysaccharide gums (e.g. hydrocolloids), and mixtures of any of the foregoing. Further examples of specific gums which may be useful in the present invention include but are not limited to acacia catechu, salai guggal, indian bodellum, copaiba gum, asafetida, cambi gum, enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, modified starch, hydroxypropyl-methylcellulose, methylcellulose, and other cellulosic materials such as sodium carboxymethylcellulose and hydroxypropyl cellulose, mixtures of any of the foregoing, and the like. This list is not meant to be exclusive.

Preferably the sustained release excipient of the present invention further comprises an inert diluent. The inert diluent of the sustained release excipient preferably comprises a pharmaceutically acceptable saccharide, including a monosaccharide, a disaccharide, or a polyhydric alcohol, and/or mixtures of any of the foregoing. Examples of suitable inert pharmaceutical fillers include sucrose, dextrose, lactose, microcrystalline cellulose, fructose, xylitol, sorbitol, mannitol, starches, other polyols, mixtures thereof and the like. However, it is preferred that a soluble pharmaceutical filler such as lactose, dextrose, mannitol, sucrose, or mixtures thereof be used. The inert diluent or filler may alternatively comprise a pre-manufactured direct compression diluent as set forth below.

In certain embodiments, the ingredients of the sustained release excipient can be pre-manufactured. In other embodiments, the active drug can be added to the sustained release excipient ingredients and that mixture wet granulated or spray granulated to form a granulation.

In certain embodiments, it is possible to dry mix the ingredients of the sustained release excipient without utilizing a wet granulation step. This procedure may be utilized, for example, where a wet granulation is to be accomplished when the active ingredient is directly added to the ingredients of the sustained release excipient. On the other hand, this procedure may also be used where no wet granulation step whatsoever is contemplated. If the mixture is to be manufactured without a wet granulation step, and the final mixture is to be tableted, it is preferred that all or part of the inert diluent comprise a pre-manufactured direct compression diluent. Such direct compression diluents are widely used in the pharmaceutical arts, and may be obtained from a wide variety of commercial sources. Examples of such pre-manufactured direct compression excipients include Emcocel® (microcrystalline cellulose, N.F.) and Emdex® (dextrates, N.F.), which are commercially available from JRS Pharma LP Patterson, N.Y.) and Tab-Fines (a number of direct-compression sugars including sucrose, fructose and dextrose). Other direct compression diluents include Anhydrous lactose (Lactose N.F., anhydrous direct tableting) from Sheffield Chemical, Union, N.J. 07083; Elcems® G-250 (powdered cellulose), N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltodextrin) from Grain Processing Corp., Muscatine, Iowa 52761; Neosorb 60® (Sorbitol, N.F., direct-compression from Roquet Corp., 645 5th Ave., New York, N.Y. 10022; Nu-Tab® (Compressible sugar, N.F.) from Ingredient Technology, Inc., Pennsauken, N.J. 081 10; Polyplasdone XL® (Crospovidone, N.F., cross-linked polyvinylpyrrolidone) from ISP, Wayne, N.J., 07470; Primojel® (Sodium starch glycolate, N.F., carboxymethyl starch) from Generichem Corp., Little Falls, N.J. 07424; Solka Floc® (Cellulose floc); Spray-dried lactose® (Lactose N.F., spray dried) from Foremost Whey Products, Baraboo, Wis. 53913 and DMV Corp., Vehgel, Holland; and Sta-Rx 1500® (Starch 1500) (Pregelatinized starch, N.F., compressible) from Colorcon, Inc., West Point, Pa. 19486.

In general, the formulation may be prepared as a directly compressible diluent, for example, by wet granulating, spray drying lactose or as a premixed direct compression diluent by art known methods. For purposes of the present invention, these specially treated inert diluents will be referred to as “directly compressible” inert diluents.

In further embodiments of the present invention, the directly compressible inert diluent which is used in conjunction with the sustained release pharmaceutical excipient of the present invention is an augmented microcrystalline cellulose as disclosed in U.S. Pat. No. 5,585,115, issued on Dec. 17, 1996, hereby incorporated by reference in its entirety. The augmented microcrystalline cellulose described therein is commercially available under the tradename “Prosolv” from JRS Pharma, Inc.

In certain embodiments, an effective amount of a pharmaceutically acceptable surfactant can also be added to the above-mentioned ingredients of the excipient, or added at the time the medicament is added, in order to increase the bioavailability of the medicament. An example of a suitable surfactant is docusate sodium in an amount of from about 1% to about 15% by weight of the solid dosage form. An especially preferred surfactant is sodium lauryl sulfate in an amount of from about 1% to about 15% by weight of the solid dosage form.

In one embodiment, the surfactant is dissolved in a suitable solvent such as water, and is thereafter added to the blended mixture of the sustained release excipient and the medicament. This allows the surfactant to wet the particles of the excipient such that when the solvent evaporates the particles of the medicament which precipitate are tiny and do not aggregate. A granulate of the medicament and the surfactant is obtained which is preferably finely and homogeneously dispersed in the excipient.

The surfactants which may be used in the present invention generally include pharmaceutically acceptable anionic surfactants, cationic surfactants, amphoteric (amphipathic/amphophilic) surfactants, and non-ionic surfactants. Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates (including sodium lauryl sulfate (SLS)), ethoxylated alkyl sulfates, ester linked sulfonates (including docusate sodium or dioctyl sodium succinate (DSS)), alpha olefin sulfonates, and phosphated ethoxylated alcohols.

Suitable pharmaceutically acceptable cationic surfactants include, for example, monoalkyl quaternary ammonium salts, dialkyl quaternary ammonium compounds, amidoamines, and aminimides.

Suitable pharmaceutically acceptable amphoteric (amphipathic/amphophilic) surfactants, include, for example, N-substituted alkyl amides, N-alkyl betaines, sulfobetaines, and N-alkyl δ-aminoproprionates.

Other suitable surfactants for use in conjunction with the present invention include polyethyleneglycols as esters or ethers. Examples include polyethoxylated castor oil, polyethoxylated hydrogenated castor oil, polyethoxylated fatty acid from castor oil or polyethoxylated fatty acid from hydrogenated castor oil. Commercially available surfactants which can be used are known under trade names Cremophor®, Myrj®, Polyoxyl 40® stearate, Emerest 2675®, Lipal 395® and PEG 3350®.

In certain embodiments of the present invention, a pH modifying agent may be included in the dosage form. When a pH modifying agent is included in the dosage form, preferably it is present from about 0.5% to about 10% by weight of the final dosage form and the pH modifying agent facilitates the release of the drug from the matrix. In certain embodiments, the pH modifying agent preferably facilitates the release of the torsemide or pharmaceutically acceptable salt thereof by the formulation to provide high bioavailability. In certain embodiments, the pH modifying agent is an acid, preferably an organic acid such as citric acid, succinic acid, fumaric acid, malic acid, maleic acid, glutaric acid, lactic acid, and the like. In certain embodiments, the pH is a base. Suitable inorganic bases include sodium hydroxide, potassium hydroxide and carbonates and bicarbonates of sodium and potassium and other suitable elements, and the like. Suitable organic bases include propanolamine, ethanolamine, methylamine, dimethyl formamide, dimethylacetamide, diethanolamine, diisopropanolamine, triethanolamine, and the like.

In certain embodiments, an ionizable gel strength enhancing agent is included in the dosage form. The ionizable gel strength enhancing agent which is optionally used in conjunction with the present invention may be monovalent or multivalent metal cations. The preferred salts are the inorganic salts, including various alkali metal and/or alkaline earth metal sulfates, chlorides, borates, bromides, citrates, acetates, lactates, etc. Specific examples of suitable ionizable gel strength enhancing agents include organic acids, calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride. Multivalent metal cations may also be utilized. However, the preferred ionizable gel strength enhancing agents are bivalent. Particularly preferred salts are calcium sulfate and sodium chloride. The ionizable gel strength enhancing agent of the present invention is added in an amount effective to obtain a desirable increased gel strength due to the cross-linking of the gelling agent (e.g., the heteropolysaccharide and homopolysaccharide gums). In alternate embodiments, the ionizable gel strength enhancing agent is included in the sustained release excipient of the present invention in an amount from about 1 to about 20% by weight of the sustained release excipient, and in an amount 0.5% to about 16% by weight of the final dosage form.

In certain embodiments, a wetting agent is included in the dosage form. Preferably the wetting agent provides for an improved bioavailability of the torsemide or pharmaceutically acceptable salt thereof. Suitable wetting agents for use in conjunction with the present invention include, for example, polyethyleneglycols as esters or ethers. Examples include polyethoxylated castor oil, polyethoxylated hydrogenated castor oil, polyethoxylated fatty acid from castor oil or polyethoxylated fatty acid from castor oil or polyethoxylated fatty acid from hydrogenated castor oil. Commercially available wetting agents which can be used are known under trade names Cremophor, Myrj, Polyoxyl 40 stearate, Emerest 2675, Lipal 395 and PEG 3350. An especially preferred wetting agent is polyethylene glycol 4000.

Preferably the wetting agent is dissolved in a suitable solvent such as water, and is thereafter added to the blended mixture of the sustained release excipient and the medicament. This allows the wetting agent to wet the particles of the excipient such that when the solvent evaporates the particles of the medicament which precipitate are tiny and do not aggregate. A granulate of the medicament and the wetting agent is obtained which is preferably finely and homogenously dispersed in the excipient. When a wetting agent is included in the dosage form, preferably the wetting agent is included in an amount from about 1% to about 20%, preferably from about 2 to about 15% of the final product, by weight.

In certain embodiments of the present invention, the sustained release excipient (e.g., matrix) of the present invention comprises a sustained release excipient which comprises from about 10 to about 99 percent by weight of a gelling agent comprising a heteropolysaccharide gum and a homopolysaccharide gum, from about 0 to about 20 percent by weight of an ionizable gel strength enhancing agent, and from about 1 to about 89 percent by weight of an inert pharmaceutical diluent. In other embodiments, the sustained release excipient comprises from about 10 to about 75 percent gelling agent, from about 2 to about 15 percent ionizable gel strength enhancing agent, and from about 30 to about 75 percent inert diluent. In yet other embodiments, the sustained release excipient comprises from about 30 to about 75 percent gelling agent, from about 5 to about 10 percent ionizable gel strength enhancing agent, and from about 15 to about 65 percent inert diluent.

The sustained release excipient of the present invention may be further modified by incorporation of a hydrophobic material which slows the hydration of at least one gum without disrupting the hydrophilic matrix when the formulation is exposed to an environmental fluid. This is accomplished in alternate embodiments of the present invention by granulating the sustained release excipient with the solution or dispersion of a hydrophobic material prior to the incorporation of the medicament. The hydrophobic polymer may be selected from an alkylcellulose such as ethylcellulose, other hydrophobic cellulosic materials, polymers or copolymers derived from acrylic or methacrylic acid esters, copolymers of acrylic and methacrylic acid esters, zein, waxes, shellac, hydrogenated vegetable oils, combinations thereof, and any other pharmaceutically acceptable hydrophobic material known to those skilled in the art. The amount of hydrophobic material incorporated into the sustained release excipient is that which is effective to slow the hydration of the gums without disrupting the hydrophilic matrix formed upon exposure to an environmental fluid. In certain preferred embodiments of the present invention, the hydrophobic material is included in the sustained release excipient in an amount from about 1 to about 20 percent by weight. The solvent for the hydrophobic material may be an aqueous or organic solvent, or mixtures thereof. Alternatively, in certain embodiments, the hydrophobic material may be coated onto the formulations of the present invention to provide for the sustained release of the formulation. In certain preferred embodiments, a hydrophobic material is included in the matrix and is coated onto the formulation.

In certain embodiments where the sustained release excipient of the present invention has been pre-manufactured, it is then possible to blend the same with the torsemide or a pharmaceutically acceptable salt thereof, e.g., in a high shear mixer.

In certain preferred embodiments of the present invention, the dosage form includes a dose of torsemide or pharmaceutically acceptable salt thereof in an amount of from about 1 to about 500 mg, from about 1 to about 400 mg, from about 2.5 mg to about 200 mg, preferably from about 5 mg to about 150 mg, more preferably from about 10 to about 110 mg. In certain preferred embodiments, the torsemide or pharmaceutically acceptable salt thereof is in an amount of from about 2.5 to about 500 mg. In certain embodiments, the torsemide or pharmaceutically acceptable salt thereof is in an amount of about 2.5, 5, 10, 20, 30, or 40, 80, 100, 110, 150, 200 mg, or 500 mg.

The sustained release excipients of the present invention preferably have uniform packing characteristics over a range of different particle size distributions and are capable of processing into the final dosage form (e.g., tablets) using either direct compression, following addition of drug and lubricant powder, or conventional wet granulation.

In certain embodiments, the properties and characteristics of a specific excipient system prepared according to the present invention is dependent in part on the individual characteristics of the homo and heteropolysaccharide constituents, in terms of polymer solubility, glass transition temperatures etc., as well as on the synergism both between different homo- and heteropolysaccharides and between the homo and heteropolysaccharides and the inert saccharide constituent(s) in modifying dissolution fluid-excipient interactions.

The combination of the gelling agent (e.g., a mixture of xanthan gum and locust bean gum) with the inert diluent, with or without the ionizable gel strength enhancing agent and hydrophobic polymer, provides a ready-to-use sustained release excipient product in which a formulator need only blend the desired active medicament, an optionally wetting agent, an optional pH modifying agent, an optional surfactant and an optional lubricant with the excipient before compressing the mixture to form slow release tablets. The excipient may comprise a physical admix of the gums along with a soluble excipient such as compressible sucrose, lactose or dextrose, although it is preferred to granulate or agglomerate the gums with plain (i.e., crystalline) sucrose, lactose, dextrose, etc., to form an excipient. The granulate form has certain advantages including the fact that it can be optimized for flow and compressibility; it can be tableted, formulated in a capsule, extruded and spheronized with an active medicament to form pellets, etc.

The pharmaceutical excipients prepared in accordance with the present invention may be prepared according to any agglomeration technique to yield an acceptable excipient product. In wet granulation techniques, the desired amounts of the heteropolysaccharide gum, the homopolysaccharide gum, and the inert diluent are mixed together and thereafter a moistening agent such as water, propylene glycol, glycerol, alcohol or the like is added to prepare a moistened mass. Next, the moistened mass is dried. The dried mass is then milled with conventional equipment into granules. Therefore, the excipient product is ready to use.

In a preferred embodiment where the sustained release excipient is pre-manufactured, the sustained release excipient is preferably free-flowing and directly compressible. Accordingly, the excipient may be mixed in the desired proportion with a therapeutically active medicament and optional lubricant (dry granulation). Alternatively, all or part of the excipient may be subjected to a wet granulation with the active ingredient and thereafter tableted. When the final product to be manufactured is tablets, the complete mixture, in an amount sufficient to make a uniform batch of tablets, is then subjected to tableting in a conventional production scale tableting machine at normal compression pressure, i.e. about 2000-1600 lbs/sq in. However, the mixture should not be compressed to such a degree that there is subsequent difficulty in its hydration when exposed to gastric fluid.

One of the limitations of direct compression as a method of tablet manufacture is the size of the tablet. If the amount of active is high a pharmaceutical formulator may choose to wet granulate the active with other excipients to attain a decent size tablet with the right compact strength. Usually the amount of filler/binder or excipients needed in wet granulation is less than that in direct compression since the process of wet granulation contributes to some extent toward the desired physical properties of a tablet.

In certain embodiments, the average particle size of the granulated excipient of the present invention ranges from about 50 microns to about 400 microns and preferably from about 185 microns to about 265 microns. The particle size of the granulation is not narrowly critical, the important parameter being that the average particle size of the granules, must permit the formation of a directly compressible excipient which forms pharmaceutically acceptable tablets. In certain embodiments, the desired tap and bulk densities of the granulation of the present invention are normally between from about 0.3 to about 0.8 g/ml, with an average density of from about 0.5 to about 0.7 g/ml. Preferably, the tablets formed from the granulations of the present invention are from about 5 to about 20 kg hardness. In certain embodiments, the average flow of the granulations prepared in accordance with the present invention are from about 25 to about 40 g/sec. Tablets compacted using an instrumented rotary tablet machine have been found to possess strength profiles which are largely independent of the inert saccharide component. Scanning electron photomicrographs of largely tablet surfaces have provided qualitative evidence of extensive plastic deformation on compaction, both at the tablet surface and across the fracture surface, and also show evidence of surface pores through which initial solvent ingress and solution egress may occur.

In further embodiments, the dosage form may be coated with a film coating e.g., a hydrophilic coating, in addition to or instead of the above-mentioned coatings. An example of a suitable material which may be used is hydroxypropylmethylcellulose (e.g., Opadry® as described above). The film coating of the present invention should be capable of producing a strong, continuous film that is smooth and elegant, capable of supporting pigments and other coating additives, non-toxic, inert, and tack-free.

Additionally, the compressed tablets may optionally be coated with a color coat that rapidly disintegrates or dissolves in water or the environment of use. The color coat may be a conventional sugar or polymeric film coating which is applied in a coating pan or by conventional spraying techniques. Preferred materials for the color coat are commercially available under the Opadry tradename (e.g, Opadry II® White). The color coat may be applied directly onto the tablet core, or may be applied after a coating as described above. Generally, the color coat surrounding the core will comprise from about 1 to about 5% preferably about 2 to about 4% based on the total weight of the tablet.

An effective amount of any generally accepted pharmaceutical lubricant or mixture of lubricants, including the calcium or magnesium soaps may be added to the above-mentioned ingredients of the formulation at the time the medicament is added, or in any event prior to compression into a solid dosage form. An example of a suitable lubricant is magnesium stearate in an amount of about 0.3% to about 3% by weight of the solid dosage form. An especially preferred lubricant is sodium stearyl fumarate, NF, commercially available under the trade name Pruv®. Other preferred lubricants include magnesium stearate and talc.

An effective amount of any generally acceptable pharmaceutical glidant or mixture of glidants may also be added to the above-mentioned ingredients of the formulation at the time the medicament is added, or in any event prior to compression into a solid dosage form. Glidants for use in the present invention include, for example, colloidal silicon dioxide, talc, silicon dioxide, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch, starch 1500, magnesium lauryl sulfate, magnesium oxide, and mixtures thereof.

In certain embodiments, additional inert diluent may also be incorporated in the sustained release oral dosage form when mixing the sustained release excipient with the torsemide or pharmaceutically acceptable salt thereof. The inert diluent may be the same or different inert diluent that is incorporated into the sustained release excipient. Other pharmaceutically acceptable diluents and excipients that may be used to formulate oral dosage forms of the present invention are described in the Handbook of Pharmaceutical Excipients, American Pharmaceutical Association (1986).

In additional embodiments of the present invention, a support platform is applied to the tablets manufactured in accordance with the present invention. Suitable support platforms are well known to those skilled in the art. An example of suitable support platforms is set forth, e.g., in U.S. Pat. No. 4,839,177, hereby incorporated by reference. In that patent, the support platform partially coats the tablet, and consists of a polymeric material insoluble in aqueous liquids. The support platform may, for example, be designed to maintain its impermeability characteristics during the transfer of the therapeutically active medicament. The support platform may be applied to the tablets, e.g., via compression coating onto part of the tablet surface, by spray coating the polymeric materials comprising the support platform onto all or part of the tablet surface, or by immersing the tablets in a solution of the polymeric materials.

The support platform may have a thickness of, e.g., about 2 mm if applied by compression, and about 10 μm if applied via spray-coating or immersion-coating. Generally, in embodiments of the invention wherein a hydrophobic polymer or enteric coating is applied to the tablets, the tablets are coated to a weight gain from about 1 to about 20%, and in certain embodiments preferably from about 5% to about 10%.

Materials useful in the hydrophobic coatings and support platforms of the present invention include derivatives of acrylic acid (such as esters of acrylic acid, methacrylic acid, and copolymers thereof) celluloses and derivatives thereof (such as ethylcellulose), polyvinylalcohols, and the like.

In certain embodiments of the present invention, the tablet core includes an additional dose of the medicament included in either the hydrophobic or enteric coating, or in an additional overcoating coated on the outer surface of the tablet core (without the hydrophobic or enteric coating) or as a second coating layer coated on the surface of the base coating comprising the hydrophobic or enteric coating material.

The coatings of the present invention may be applied in any pharmaceutically acceptable manner known to those skilled in the art. For example, in one embodiment, the coating is applied via a fluidized bed or in a coating pan. The solvent for the hydrophobic polymer or enteric coating may be organic, aqueous, or a mixture of an organic and an aqueous solvent. The organic solvents may be, e.g., isopropyl alcohol, ethanol, and the like, with or without water.

In certain preferred embodiments of the present invention, the sustained release dosage form includes an immediate release component which comprises an effective amount of torsemide or pharmaceutically acceptable salt thereof. In such embodiments, an effective amount of the torsemide in immediate release form may be coated onto the multiparticulates or tablets of the present invention. For example, where the extended release torsemide from the formulation is due to a controlled release coating, the immediate release layer would be overcoated on top of the controlled release coating. On the other hand, the immediate release layer may be coated onto the surface of multiparticulates or tablets wherein the torsemide is incorporated in a controlled release matrix. Where a plurality of the sustained release multiparticulates comprising an effective unit dose of the torsemide or pharmaceutically acceptable salt thereof are incorporated into a capsule, the immediate release portion of the torsemide dose may be incorporated into the capsule via inclusion of a sufficient amount of immediate release torsemide as a powder or granulate within the capsule. Alternatively, the capsule itself may be coated with an immediate release layer of the torsemide.

In preferred embodiments, wherein the oral dosage form includes the torsemide or pharmaceutically acceptable salt thereof in immediate release component, the oral dosage form is in the form of a bilayer tablet including a sustained release portion and an immediate release portion. Preferably the immediate release portion comprises torsemide or a pharmaceutically acceptable salt thereof in combination with an immediate release excipient which may include any of the ingredients described herein with respect to the sustained release oral dosage form, however, the ingredients are in an amount which allows for the immediate release of the torsemide or pharmaceutically acceptable salt thereof upon exposure to an environmental fluid. For example, in certain embodiments, the immediate release portion of the bilayer oral dosage form may optionally include a gelling agent as described herein, a pharmaceutically acceptable diluent such as microcrystalline cellulose, and other pharmaceutically acceptable excipients described above (e.g., lubricant, diluent, wetting agent, pH modifying agent, surfactants, and the like), in an amount such that the torsemide is able to release in an immediate release manner from the dosage form.

In certain preferred embodiments, the present invention is further directed to a method for preparing a sustained release bilayer dosage form, comprising preparing a first layer comprising a sustained release excipient comprising a gelling agent, ionizable gel strength enhancing agent, and pharmaceutically acceptable inert diluent. Thereafter a granulation solution optionally comprising a wetting agent and pH-modifying agent is added to the first portion of sustained release excipient and granulated. The granulation is then dried and milled. An optional glidant is added to the blend. Thereafter, an opitional lubricant is added. The second layer of the bilayer dosage form is prepared by combining an immediate release excipient optionally comprising a gelling agent, optionally an ionizable gel strength enhancing agent, and a pharmaceutically acceptable inert diluent with an effective amount of torsemide. Thereafter, an optional glidant is added and blended. An optional lubricant is then added and blended. The two layers are dispensed into separate hoppers of a bilayer tablet press and compressed.

The inclusion of an immediate release form of torsemide or pharmaceutically acceptable salt thereof may be desired when, for example, a loading dose of a therapeutically active agent is needed to provide therapeutically effective blood levels of the active agent when the formulation is first exposed to gastric fluid. The loading dose of medicament included in the coating layer, the immediate release layer of the bilayer dosage form may be, e.g., from about 10% to about 40% of the total amount of medicament included in the formulation.

One skilled in the art would recognize still other alternative manners of incorporating the immediate release torsemide portion into the unit dose. Such alternatives are deemed to be encompassed by the appended claims.

In certain embodiments, a second therapeutically effective agent is included in the sustained release oral dosage forms of the present invention. Preferably, the second therapeutic agent is also useful for the treatment of edema. Such secondary drugs include for example and without limitation anti-hypertensive agents (e.g., ACE-inhibitors, calcium channel blockers, alpha-adrenergic blockers, beta-adrenergic blockers, and the like), other diuretics (e.g., loop-diuretics, thiazide diuretics, potassium sparing diuretics), digitalis glucosides, organic nitrates, combinations thereof, and the like. The second agent may be included in sustained release form or in immediate release form. In certain embodiments, the secondary drug is incorporated into the sustained release matrix along with the torsemide or a pharmaceutically acceptable salt thereof, is incorporated as a powder, granulation, etc. in the dosage form, or is incorporated into the sustained release oral dosage form in a coating on the dosage form.

### DETAILED DESCRIPTION OF THE PREFERRED EMBODIMENTS

The following examples illustrate various aspects of the present invention. They are not to be construed to limit the claims in any manner whatsoever.

### EXAMPLES 1-2

In Examples 1 and 2, sustained release excipients in accordance with the present invention were prepared. The sustained release excipient was prepared by dry blending the requisite amounts of xanthan gum, locust bean gum, calcium sulfate and mannitol in a high speed mixer/granulator. While running choppers/impellers, water was added to the dry blended mixture, and granulated. The granulation was then dried in a fluid bed dryer to a LOD (loss on drying) of less than about 10% by weight (e.g., 4-7% LOD). The granulation was then milled using comminuting machine. The ingredients of the sustained release excipient of Examples 1 and 2 are set forth in Table 1 below:

TABLE 1

| | Amount (50% gum) | Amount (70% gum) |
|---|---|---|
| Component | Example 1 | Example 2 |
| Xanthan Gum | 20% | 28% |
| Locust Bean Gum | 30% | 42% |
| Calcium Sulfate Dihydrate | 10% | 10% |
| Mannitol, USP | 40% | 20% |
| Water | q.s. | q.s. |

*Removed during processing

### EXAMPLES 3-6

To study the effect of active:gum ratio, different percentages of the sustained release excipient from Example 1 prepared as described above were dry blended with a desired amount of torsemide. A suitable amount of tableting glidant and lubricant, silicon dioxide and magnesium stearate, NF, respectively, were added, and the mixture was blended. The final mixture was compressed into tablets, each tablet containing 100 mg torsemide (Ex.3-Ex.6). Tablets were compressed at a hardness of 2-8 Kp. The tablets prepared in accordance with Examples 3-6 are listed in Table 2 below:

TABLE 2

| | Amount mg/tablet (%/tablet)* | | | | | | | |
|---|---|---|---|---|---|---|---|---|
| Component | Ex. 3 | | Ex. 4 | | Ex. 5 | | Ex. 6 | |
| Sustained Release Excipient (50%) | 100 | (49.0) | 200 | (65.4) | 300 | (73.5) | 400 | (78.4) |
| Torsemide | 100 | (49.0) | 100 | (32.7) | 100 | (24.5) | 100 | (19.6) |
| Silicon dioxide | 2 | (1.0) | 3 | (1.0) | 4 | (1.0) | 5 | (1.0) |
| Magnesium Stearate | 2 | (1.0) | 3 | (1.0) | 4 | (1.0) | 5 | (1.0) |
| Total Weight (mg/tablet) | 204 | | 306 | | 408 | | 510 | |
| Active:Gum Ratio | 1:0.5 | | 1:1 | | 1:1.5 | | 1:2 | |

*Percentage by weight of the dosage form is indicated in parenthesis

The tablets prepared in accordance with Examples 3-6 were dissolution tested in USP 26(2003) dissolutiin Apparatus type III, at pH change media with an agitation of 15 dpm. The volume and temperature for the media were 250 ml and 37° C., respectively. The tablets were tested at 0, 1, 3, 7, 12, 16, and 24 time points. The dissolution results are listed in Table 2A below.

TABLE 2A

| Time (h) | Ex. 3 | Ex. 4 | Ex. 5 | Ex. 6 |
|---|---|---|---|---|
| 0 | 0 | 0 | 0 | 0 |
| 1 | 43.5 | 12.9 | 6.8 | 5.7 |
| 3 | 45.3 | 14.6 | 7.5 | 6.5 |
| 7 | 51.6 | 24.2 | 12.1 | 11.7 |
| 12 | 65.9 | 41.4 | 24.0 | 21.6 |
| 16 | 75.2 | 64.6 | 32.4 | 30.4 |
| 24 | 95.6 | 90.7 | 43.7 | 42.5 |
| % Recovery | 100.0 | 100.0 | 100.0 | 100.0 |

Conclusion: As shown in Ex.3-Ex.6, the dissolution rate was inversely proportional to the amount of sustained release excipient present in the formulation. There was slight difference in dissolution rates between the formulation made with 73% (Ex.5) and 78.4% (Ex.6).

### EXAMPLES 7-12

To study the effect of a wetting agent and/or pH modifying agent, the sustained release excipient prepared in accordance with Example 1 and a desired amount of torsemide was dry blended in a mixer or granulator. While running the impellers, the wetting agent and/or pH modifying agent solution was added slowly to the dry blended mixture, and granulated. The granulation was then dried in a room temperature or a fluid bed dryer to a LOD (loss on drying) of less than about 4%. The granulation was then screened through a #20 mesh screen or milled through a Fitzmill. The screened or milled granulation was then blended with a suitable amount of tableting glidant and lubricant, silicon dioxide and magnesium stearate, NF, respectively. This final mixture was compressed into tablets, each tablet containing 100 mg torsemide (Ex. 7-12 below). Tablets were compressed at a hardness of 6-16 Kp. The formulations prepared with the wetting and/or pH modifying agents are listed as Examples 7-12 in Tables 3, 4, & 5 below:

### Effect of Wetting Agent

TABLE 3

| | Amount mg/tablet (%/tablet)** | | | |
|---|---|---|---|---|
| Component | Ex. 7 | | Ex. 8 | |
| Sustained Release Excipient (50%) | 200 | (62.3) | 200 | (59.5) |
| Torsemide | 100 | (31.2) | 100 | (29.8) |
| Polyethylene glycol 4000 | 15 | (4.7) | 30 | (8.9) |
| (wetting agent) | | | | |
| Silicon dioxide | 3 | (0.9) | 3 | (0.9) |
| Magnesium Stearate | 3 | (0.9) | 3 | (0.9) |
| Total Weight (mg/tablet) | 321 | | 336 | |
| Active:Gum Ratio | 1:1 | | 1:1 | |
| Water* | q.s. | | q.s. | |

*Removed during processing
**Percentage by weight of the dosage form is indicated in parenthesis

The tablets prepared in accordance with Examples 7-8 were dissolution tested in dissolution test and parameters of Examples 3-6. The dissolution results for Examples 7-8 are listed in Table 3A below.

TABLE 3A

| Time (h) | Ex. 7 | Ex. 8 |
|---|---|---|
| 0 | 0 | 0 |
| 1 | 8.0 | 6.7 |
| 3 | 9.7 | 7.6 |
| 7 | 26.1 | 29.7 |
| 12 | 53.9 | 64.5 |
| 16 | 73.0 | 85.2 |
| 24 | 100.0 | 100.0 |
| % Recovery | 100.0 | 100.0 |

Conclusion: As indicated in Table 3A, the dissolution rate from the formulation containing 8.9% of wettin agent (Ex.8) was faster (64.5% versus 53.9%) at 12 hours than the rate from the formultion containing 4.7% (Ex.7) wetting agent.

### Effect of pH Modifying Agent

TABLE 4

| | Amount mg/tablet (%/tablet)** | | | |
|---|---|---|---|---|
| Component | Ex. 9 | | Ex. 10 | |
| Sustained Release Excipient (50%) | 200 | (64.7) | 200 | (64.1) |
| Torsemide | 100 | (32.4) | 100 | (32.1) |
| KOH (pH modifying agent) | 3 | (1.0) | 6 | (1.9) |
| Silicon dioxide | 3 | (1.0) | 3 | (1.0) |
| Magnesium Stearate | 3 | (1.0) | 3 | (1.0) |
| Total Weight (mg/tablet) | 309 | | 312 | |
| Active:Gum Ratio | 1:1 | | 1:1 | |
| Water* | q.s. | | q.s. | |

*Removed during processing
**Percentage by weight of the dosage form is indicated in parenthesis

The tablets prepared in accordance with Examples 9-10 were dissolution tested in dissolution test and parameters of Examples 3-6. The dissolution results for Examples 9-10 are listed in Table 4A below.

TABLE 4A

| Time (h) | Ex. 9 | Ex. 10 |
|---|---|---|
| 0 | 0 | 0 |
| 1 | 7.0 | 5.7 |
| 3 | 8.6 | 6.3 |
| 7 | 20.8 | 11.3 |
| 12 | 49.1 | 46.0 |
| 16 | 67.9 | 67.3 |
| 24 | 100.0 | 96.5 |
| % Recovery | 100.0 | 100.0 |

Conclusion: As shown in Table 4A, the dissolution rate from the formulation containing ˜2% of pH modifying agent (Ex.10) was slower (11.3% versus 20.8%) at 7 hours, but slightly different at the other time points than the rate from the formulation containing 1% wetting agent (Ex.9).

### Effect of a combination of Wetting Agent and pH Modifying Agent

TABLE 5

| | Amount mg/tablet (%/tablet)** | | | |
|---|---|---|---|---|
| Component | Ex. 11 | | Ex. 12 | |
| Sustained Release Excipient (50%) | 200 | (61.7) | 200 | (58.5) |
| Torsemide | 100 | (30.9) | 100 | (29.2) |
| KOH (pH modifying agent) | 3 | (0.9) | 6 | (1.8) |
| Polyethylene glycol 4000 | 15 | (4.6) | 30 | (8.8) |
| (wetting agent) | | | | |
| Silicon dioxide | 3 | (0.9) | 3 | (0.9) |
| Magnesium Stearate | 3 | (0.9) | 3 | (0.9) |
| Total Weight (mg/tablet) | 324 | | 342 | |
| Active:Gum Ratio | 1:1 | | 1:1 | |
| Water* | q.s. | | q.s. | |

*Removed during processing
**Percentage by weight of the dosage form is indicated in parenthesis

The tablets prepared in accordance with Examples 11-12 were dissolution tested in dissolution test and parameters of Examples 3-6. The dissolution results for Examples 11-12 are listed in Table 5A below.

TABLE 5A

| Time (h) | Ex. 11 | Ex. 12 |
|---|---|---|
| 0 | 0 | 0 |
| 1 | 7.5 | 7.9 |
| 3 | 9.5 | 8.8 |
| 7 | 24.5 | 33.0 |
| 12 | 54.7 | 76.5 |
| 16 | 79.3 | 96.3 |
| 24 | 100.0 | 100.0 |
| % Recovery | 100.0 | 100.0 |

Conclusion: As indicated in Table 5A, the dissolution rate from the formulation containing a combination of ˜9% wetting agent and ˜2% of pH modifying agent (Ex.12) was faster (76.5% versus 54.7%) at 12 hours than the rate from the formulation containing a combination of 4.6% wetting agent and ˜1% pH modifying agent (Ex.11).

### EXAMPLES 13-16

The Examples 13-16, formulations having different dosages of torsemide were prepared. The sustained release excipient prepared in accordance with Example 1 was dry blended with a desired amount of torsemide. The wetting agent and pH modifying agent solution was added slowly to the dry blended mixture, and granulated. The granulation was then dried to a LOD (loss on drying) of less then about 4%. The granulation was then passed through #20 mesh screen or milled through a Fitzmill. The screened or milled granulation is then blended with a suitable amount of tableting glidant and lubricant, silicon dioxide and magnesium stearate, NF, respectively. This final mixture was compressed into tablets, each tablet containing between 40-200 mg toresmide (Ex.13-Ex.16). Tablets were compressed at a hardness of 6-16 Kp. The different dose formulations prepared are listed in Table 6 below:

TABLE 6

| | Amount mg/tablet (%/tablet)** | | | | | | | |
|---|---|---|---|---|---|---|---|---|
| Component | Ex. 13 | | Ex. 14 | | Ex. 15 | | Ex. 16 | |
| Sustained Release Excipient (50%) | 80 | (56.8) | 150 | (56.8) | 200 | (58.5) | 400 | (58.5) |
| Torsemide | 40 | (28.4) | 75 | (28.4) | 100 | (29.2) | 200 | (29.2) |
| KOH (pH modifying agent) | 3.2 | (2.3) | 6 | (2.3) | 6 | (1.8) | 12 | (1.8) |
| Polyethylene glycol 4000 (wetting agent) | 16 | (11.4) | 30 | (11.4) | 30 | (8.8) | 60 | (8.8) |
| Silicon dioxide | 0.8 | (0.6) | 1.5 | (0.6) | 3 | (0.9) | 6 | (0.9) |
| Magnesium Stearate | 0.8 | (0.6) | 1.5 | (0.6) | 3 | (0.9) | 6 | (0.9) |
| Total Weight (mg/tablet) | 140.8 | | 264 | | 342 | | 684 | |
| Active:Gum Ratio | 1:1 | | 1:1 | | 1:1 | | 1:1 | |
| Water* | q.s. | | q.s. | | q.s. | | q.s. | |

*Removed during processing
**Percentage by weight of the dosage form is indicated in parenthesis

The tablets prepared in accordance with Examples 13-15 were dissolution tested in dissolution test and parameters of Examples 3-6. The dissolution results for Examples 13-15 are listed in Table 6A below.

TABLE 6A

| Time (h) | Ex. 13 | Ex. 14 | Ex. 15 |
|---|---|---|---|
| 0 | 0 | 0 | 0 |
| 1 | 14.6 | 31.6 | 8.2 |
| 3 | 17.1 | 33.7 | 9.7 |
| 7 | 41.8 | 53.7 | 29.6 |
| 12 | 91.0 | 87.2 | 73.6 |
| 16 | 100.0 | 99.6 | 92.9 |
| 24 | 100.0 | 100.0 | 100.0 |
| % Recovery | 100.0 | 100.0 | 100.0 |

Conclusion: As indicated in Table 6A, the different formulations provided varied dissolution rates.

### EXAMPLES 17-19

In Example 17-19, the sustained release excipient prepared in accordance with Example 1 and hydrophobic polymer (Acrylic Copolymer Eudragit RS PO and/or Eudragit RL PO)) were dry blended with a desired amount of torsemide or a pharmaceutically acceptable salt thereof in a granulator. The wetting agent/pH modifying agent solution was added slowly to the dry blended mixture, and granulated. The granulation was then dried in a Fluid Bed Drier to a LOD (loss on drying) of less than about 4%. The granulation was then milled through a Fitzmill. The milled granulation was then blended with a suitable amount of tableting glidant and lubricant, silicon dioxide and magnesium stearate, NF, respectively. This final mixture was compressed into tablets, each tablet containing 100 mg torsemide. Tablets were compressed at a hardness of 4-12 Kp. The formulations prepared in accordance with Examples 17-19 are listed in Table 7 below:

### Effect of Hydrophobic Polymer

TABLE 7

| | Amount mg/tablet (%/tablet)** | | | | | |
|---|---|---|---|---|---|---|
| Component | Ex. 17 | | Ex. 18 | | Ex. 19 | |
| Sustained Release | 100 | (36.0) | 100 | (36.0) | 100 | (36.0) |
| Excipient (50%) | | | | | | |
| Torsemide | 100 | (36.0) | 100 | (36.0) | 100 | (36.0) |
| Eudragit RS PO | 50 | (18.0) | N/A | | 40 | (14.4) |
| (hydrophobic polymer) | | | | | | |
| Eudragit RL PO | N/A | | 50 | (18.0) | 10 | (3.6) |
| (hydrophobic polymer) | | | | | | |
| KOH (pH modifying | 5 | (1.8) | 5 | (1.8) | 5 | (1.8) |
| agent) | | | | | | |
| Polyethylene glycol | 20 | (7.2) | 20 | (7.2) | 20 | (7.2) |
| 4000 (wetting agent) | | | | | | |
| Silicon dioxide | 1.5 | (0.5) | 1.5 | (0.5) | 1.5 | (0.5) |
| Magnesium Stearate | 1.5 | (0.5) | 1.5 | (0.5) | 1.5 | (0.5) |
| Total Weight (mg/ | 278 | | 278 | | 278 | |
| tablet) | | | | | | |
| Active:Gum Ratio | 1:0.5 | | 1:0.5 | | 1:0.5 | |
| Water* | q.s. | | q.s. | | q.s. | |

*Is removed during processing
**Percentage by weight of the dosage form is indicated in parenthesis

The tablets prepared in accordance with Examples 17-19 were dissolution tested in dissolution test and parameters Examples 3-6. The dissolution results for Examples 17-19 are listed in Table 7A below.

TABLE 7A

| Time (h) | Ex. 17 | Ex. 18 | Ex. 19 |
|---|---|---|---|
| 0 | 0 | 0 | 0 |
| 1 | 12.3 | 20.1 | 17.1 |
| 3 | 14.8 | 23.6 | 21.5 |
| 7 | 30.2 | 38.3 | 39.8 |
| 12 | 51.2 | 59.6 | 63.2 |
| 16 | 67.1 | 73.4 | 81.0 |
| 24 | 90.4 | 95.9 | 98.1 |
| % Recovery | 100.0 | 100.0 | 100.0 |

Conclusion: As shown in Table 7A, the dissolution rate from the formulation containing 18% Eudragit RD PO (hydrophobic polymer), Ex.17, was slower (51.2% versus 59.6%) at 12 hours than the rated from the formulaton contining the same percentage of Eudragit RL PO (hydrophobic polymer), Ex.18. The dissolution rate from the formulation of Ex. 18, was slightly different than the rate from the formulation of Ex. 19 containing a combination of 14.4% Eudragit RS PO and 3.6% Eudragit RL PO.

### EXAMPLE 20

In Example 20, a bilayer tablet formulation was prepared. The ingredients of the formulation of Example 20 are set forth in Table 8 below:

TABLE 8

| | Amount | Amount |
|---|---|---|
| Component | mg/tablet | % w/w |
| Part A (sustained release portion) | | |
| Sustained Release Excipient of Example 1 | 200 | 43.5 |
| Torsemide | 75 | 16.3 |
| Polyethylene glycol (wetting agent) | 23 | 5.0 |
| KOH (pH modifying agent) | 6 | 1.3 |
| Silicon dioxide | 2 | 0.4 |
| magnesium stearate | 2 | 0.4 |
| Total (controlled release portion) | 308 | 66.9 |
| Part B (immediate release portion) | | |
| Torsemide | 25 | 5.4 |
| Sustained Release Excipient of Example 1 | 25 | 5.4 |
| MCC | 100 | 21.7 |
| Silicon dioxide | 1 | 0.2 |
| magnesium stearate | 1 | 0.2 |
| Total (immediate release portion) | 152 | 32.9 |
| Total tablet weight | 460 | 100 |
| Sterile water for injection, USP* | | 10-30 |
| Active:Gum Ratio | 1:1 | 1:1 |

*removed during processing

The formulation of Example 20 was prepared as follows:

Part A—Sustained Release Portion
1. Accurately weigh all the ingredients.
2. Prepare a granulation solution by dispensing Polyethylene Glycol (PEG) into 120 g of water and then add Potassium Hydroxide, stirring until a clear solution forms.
3. Dispense the sustained release excipient (Part A) and Torsemide into a high shear granulator and mix.
4. While mixing in the high shear granulator add the granulation solution from step 2 to step 3
5. Check the granulation as the addition of water or mixing may be needed to form proper granules.
6. Dry the granulation from step 5 in a fluid bed dryer to the target LOD.
7. Mill the dried material from step 6.
8. Dispense the milled material from step 7 into V-blender.
9. Add Silicon Dioxide and blend.
10. Add Magnesium Stearate to step 9, and blend.
11. The Controlled Release portion is completed.

Part B—Immediate Release Portion
12. Accurately weigh all the ingredients.
13. Dispense the sustained release excipient (Part B), Torsemide(Part B), and Microcrystalline Cellulose (Part B) into a V-blender and blend.
14. Add Silicon Dioxide (Part B) to step 13 and blend.
15. Add Magnesium Stearate (Part B) to step 14 and blend.

Part A+B/Bilayer Tablet
16. Dispense finished blends Part A and Part B into separate hoppers of a bilayer tablet press.
17. Adjust weights of each layer to target values and compress.

### EXAMPLE 21

In Example 21, a sustained release oral dosage form was prepared. The ingredients of the formulation of Example 21 are set forth in Table 9 below:

TABLE 9

| | Amount | Amount |
|---|---|---|
| Component | mg/tablet | % w/w |
| Sustained Release Excipient of Example 1 | 100 | 34.0 |
| (Part I) | | |
| Torsemide | 100 | 34.0 |
| Eudragit RS PO (hydrophobic polymer) | 50 | 17.0 |
| Polyethylene glycol 4000 (wetting agent) | 20 | 6.8 |
| KOH (pH modifying agent) | 5 | 1.7 |
| Silicon dioxide | 2 | 0.7 |
| magnesium stearate | 2 | 0.7 |
| Sustained Release Excipient of Example 1 | 15 | 5.1 |
| (Part II) | | |
| Total tablet weight | 294 | 100 |
| Sterile water for injection, USP* | | 15-30 |
| Active:Gum Ratio | 1:0.5 | 1:0.5 |

*removed during processing

The formulation of Example 21 was prepared as follows:
1. Accurately weigh all the ingredients.
2. Prepare a granulation solution by dispensing Polyethylene Glycol (PEG) into 120 g of water and then add Potassium Hydroxide, stirring until clear solution forms.
3. Dispense sustained release excipient (Part I), Eudragit RS PO and Torsemide into a high shear granulator and mix.
4. While mixing in the high shear granulator add the granulation solution from step 2 to step 3
5. Check the granulation, as additional water or mixing may be needed to form proper granules.
6. Dry the granulation from step 5 in fluid bed dryer to the target LOD.
7. Mill the dried material from step 6.
8. Dispense the milled material from step 7 into a V-blender, add sustained release excipient (Part II) and blend.
9. Add Silicon Dioxide to step 9, and blend.
10. Add Magnesium Stearate to step 10, and blend.
11. Compress blend to target tablet weight.

### EXAMPLE 22

In Example 22, a sustained release oral dosage form was prepared. The ingredients of the formulation of Example 22 are set forth in Table 10 below:

TABLE 10

| Component | Amount mg/tablet | Amount % w/w |
|---|---|---|
| Sustained Release Excipient | 200 | 59.3 |
| Torsemide | 100 | 29.7 |
| Polyethylene glycol (wetting agent) | 25 | 7.4 |
| KOH (pH modifying agent) | 6 | 1.8 |
| Silicon dioxide | 3 | 0.9 |
| magnesium stearate | 3 | 0.9 |
| Total tablet weight | 337 | 100 |
| Sterile water for injection, USP* | | 10-30 |
| Active:Gum Ratio | 1:1 | 1:1 |

*Removed during processing

The formulation of Example 22 was prepared as follows:
1. Accurately weigh all the ingredients.
2. Prepare a granulation solution by dispensing Polyethylene Glycol (PEG) into 120 g of water and then adding Potassium Hydroxide, stirring until a clear solution forms.
3. Dispense the sustained release excipient and Torsemide into a high shear granulator and mix.
4. While mixing in the high shear granulator add the granulation solution from step 2 to step 3.
5. Check the granulation as additional water or mixing may be needed to form proper granules.
6. Dry the granulation from step 5 in a fluid bed dryer to the target LOD.
7. Mill the dried material from step 6.
8. Dispense the milled material from step 7 into a V-blender.
9. Add Silicon Dioxide to the milled material in step 8 and blend.
10. Add Magnesium Stearate to step 9, and blend.
11. Compress the blend of step 10 to target tablet weight.

### EXAMPLE 23

The tablets prepared in accordance with Examples 20-22 were dissolution tested in dissolution test and parameters of Examples 3-6. The dissolution results for Examples 20-22 are listed in Table 11 :

TABLE 11

| Time (h) | Ex. 20 | Ex. 21 | Ex. 22 |
|---|---|---|---|
| 0 | 0 | 0 | 0 |
| 1 | 31 | 11.8 | 6.9 |
| 3 | 33.8 | 15.8 | 9.5 |
| 7 | 53.2 | 40.1 | 35.7 |
| 12 | 82.5 | 75 | 76.7 |
| 16 | 94.8 | 96.6 | 95.6 |
| 24 | 100.5 | 99.7 | 101.6 |
| % Recovery | 100.5 | 99.7 | 101.6 |

### EXAMPLE 24

A single-dose, randomized, open-label, four-way cross-over pharmacokinetic study of torsemide was preformed, for the sustained release oral dosage forms prepared in accordance with Example 20-23 and one immediate release reference formulation, (Demadex® 100 mg manufactured by Roche). The formulations were administered to healthy female & male volunteers under fasting or fed conditions. Subjects were dosed with 100 mg of the three extended release formulations and 100 mg of the immediate release reference formulation in the first two dosing periods, which was subsequently reduced to a half tablet of the 100 mg dos (50 mg) in the last two periods of the study due to the occurrence of adverse events. The study was designed to be carried out in two groups namely, male group (12+4 subjects) and female group (12+4 subjects) under fasting or fed conditions. However, due to adverse events, females were not continued in the study after the first dosing period and their data was not included in the pharmacokinetic analysis. The results for the half tablet of the 100 mg dose (50 mg) of Demadex® were dose normalized to 100 mg

Blood samples were obtained pre-dose and at 0.25, 0.5, 0.75, 1.0, 1.5, 2.0, 3.0, 4.0, 6.0, 8.0, 10.0, 12.0, 14.0, 16.0, 20.0 and 24.0 hours post-dose.

Urine collections were obtained at 0-4, 4-8, 8-12, 12-16, 16-20 and 20-24 hours. A specimen prior to dosing was also obtained.

The following pharmacokinetic parameters in Tables 12 and 13 were obtained under fasting conditions:

TABLE 12

| | Example 22 | | | EXAMPLE 20 | | |
|---|---|---|---|---|---|---|
| | | | AUC₍₀₋₂₄₎ | | | AUC₍₀₋₂₄₎ |
| | Cmax | Tmax | (μg · | Cmax | Tmax | (μg · |
| | (μg/mL) | (h) | h/mL) | (μg/mL) | (h) | h/mL) |
| Mean | 1.662 | 5.13 | 13.976 | 3.948 | 1.72 | 22.563 |
| Geo. | 1.416 | 3.35 | 13.660 | 3.878 | 1.22 | 21.359 |
| Mean | | | | | | |
| Median | 1.381 | 2.50 | 13.110 | 3.897 | 1.25 | 18.539 |
| Minimum | 0.575 | 1.50 | 10.331 | 2.994 | 0.5 | 15.709 |
| Maximum | 3.364 | 14.00 | 19.382 | 4.995 | 6 | 33.37 |
| S.D. | 1.00 | 5.51 | 3.24 | 0.8 | 1.81 | 7.52 |
| % CV | 60.3 | 107.5 | 23.2 | 20.3 | 105.3 | 33.3 |
| N | 8 | 8 | 8 | 8 | 8 | 8 |

TABLE 13

| | Example 21 | | | Demadex ® 100 mg tablets | | |
|---|---|---|---|---|---|---|
| | Cmax | Tmax | AUC₍₀₋₂₄₎ | Cmax | Tmax | AUC₍₀₋₂₄₎ |
| | (μg/mL) | (h) | (μg · h/mL) | (μg/mL) | (h) | (μg · h/mL) |
| Mean | 3.364 | 4.57 | 21.506 | 14.752 | 0.86 | 34.207 |
| Geo. | 2.352 | 4.38 | 19.351 | 13.931 | 0.76 | 33.339 |
| Mean | | | | | | |
| Median | 1.538 | 4 | 16.093 | 12.452 | 0.75 | 31.882 |
| Minimum | 1.086 | 3 | 12.008 | 8.892 | 0.5 | 21.92 |
| Maximum | 10.163 | 6 | 46.72 | 22.968 | 2 | 44.915 |
| S.D. | 3.42 | 1.4 | 12.17 | 5.58 | 0.54 | 8.11 |
| % CV | 101.6 | 30.6 | 56.6 | 37.8 | 62.7 | 23.7 |
| N | 7 | 7 | 7 | 7 | 7 | 8 |

The following pharmacokinetic parameters in Tables 14 and 15 were obtained under fed conditions:

TABLE 14

| | EXAMPLE 22 | | | EXAMPLE 20 | | |
|---|---|---|---|---|---|---|
| | Cmax | Tmax | AUC₍₀₋₂₄₎ | Cmax | Tmax | AUC₍₀₋₂₄₎ |
| | (μg/mL) | (h) | (μg · h/mL) | (μg/mL) | (h) | (μg · h/mL) |
| Mean | 4.800 | 5.67 | 31.651 | 4.698 | 5.19 | 34.075 |
| Geo. | 4.254 | — | 27.443 | 4.309 | 4.47 | 31.452 |
| Mean | | | | | | |
| Median | 5.417 | 6.00 | 37.822 | 3.726 | 6 | 30.325 |
| Minimum | 1.234 | 0.00 | 9.671 | 2.557 | 1.5 | 16.478 |
| Maximum | 6.383 | 10.00 | 44.859 | 7.93 | 10 | 62.841 |
| S.D. | 1.93 | 3.44 | 15.15 | 2.11 | 2.69 | 14.76 |
| % CV | 40.1 | 60.8 | 47.9 | 44.9 | 51.9 | 43.3 |
| N | 6 | 6 | 6 | 8 | 8 | 8 |

TABLE 15

| | Example 21 | | | Demadex ® 100 mg tablets | | |
|---|---|---|---|---|---|---|
| | Cmax | Tmax | AUC₍₀₋₂₄₎ | Cmax | Tmax | AUC₍₀₋₂₄₎ |
| | (μg/mL) | (h) | (μg · h/mL) | (μg/mL) | (h) | (μg · h/mL) |
| Mean | 6.111 | 4.83 | 33.471 | 12.152 | 1.75 | 39.125 |
| Geo. | 4.485 | 4.45 | 26.528 | 11.754 | 1.67 | 36.881 |
| Mean | | | | | | |
| Median | 5.197 | 6 | 23.957 | 10.811 | 2 | 34.169 |
| Minimum | 1.096 | 2 | 12.431 | 8.982 | 0.75 | 23.514 |
| Maximum | 11.911 | 6 | 72.949 | 19.532 | 2 | 68.409 |
| S.D. | 4.52 | 1.83 | 24.95 | 3.66 | 0.48 | 15.24 |
| % CV | 73.9 | 38 | 74.5 | 30.1 | 27.4 | 39 |
| N | 6 | 6 | 6 | 7 | 7 | 7 |

The following urine excretion rates of torsemide in Tables 16 and 17 were exhibited under fasting conditions:

TABLE 16

| | EXAMPLE 22 | | | EXAMPLE 20 | | |
|---|---|---|---|---|---|---|
| Time | Excretion Rate (μg/hr) | | | Excretion Rate (μg/hr) | | |
| (hrs.) | Minimum | Mean | Maximum | Minimum | Mean | Maximum |
| 0-4 | 90.6 | 263.6 | 389.4 | 280.5 | 847.2 | 1975.6 |
| 4-8 | 39 | 290 | 649.5 | 159.7 | 346.6 | 561.4 |
| 8-12 | 83.7 | 161.2 | 255.3 | 75.6 | 234.3 | 432.6 |
| 12-16 | 69.9 | 155.7 | 289.4 | 48.4 | 174.8 | 384.6 |
| 16-20 | 66 | 220.9 | 468.6 | 57.9 | 206.4 | 563.2 |
| 20-24 | 118.3 | 181.1 | 269.7 | 14.5 | 142.9 | 345.2 |

TABLE 17

| | EXAMPLE 21 | | | Demadex ® 100 mg tablets | | |
|---|---|---|---|---|---|---|
| Time | Excretion Rate (μg/hr) | | | Excretion Rate (μg/hr) | | |
| (hrs.) | Minimum | Mean | Maximum | Minimum | Mean | Maximum |
| 0-4 | 188.7 | 277.9 | 360.7 | 1149.1 | 2603.4 | 5688.8 |
| 4-8 | 129.7 | 685.9 | 2272.7 | 290.2 | 510.6 | 1139.2 |
| 8-12 | 104 | 289.2 | 555 | 41.9 | 244.6 | 541.2 |
| 12-16 | 35.2 | 205.8 | 388.4 | 21.75 | 69.4 | 120.4 |
| 16-20 | 87.2 | 320.4 | 603.8 | 37.63 | 86.6 | 176.7 |
| 20-24 | 72 | 117 | 220.7 | 19.5 | 50.9 | 133.9 |

The following urine excretion rates of torsemide in Tables 18 and 19 were exhibited under fed conditions:

TABLE 18

| | EXAMPLE 22 | | | EXAMPLE 20 | | |
|---|---|---|---|---|---|---|
| Time | Excretion Rate (μg/hr) | | | Excretion Rate (μg/hr) | | |
| (hrs.) | Minimum | Mean | Maximum | Minimum | Mean | Maximum |
| 0-4 | 78.2 | 210.2 | 309.8 | 223.6 | 729.1 | 1386.1 |
| 4-8 | 131.6 | 1032.2 | 2886.9 | 242.5 | 857.3 | 2379.1 |
| 8-12 | 83 | 716 | 1455.2 | 49.9 | 777.7 | 1451.5 |
| 12-16 | 53.6 | 282.7 | 766.7 | 36.9 | 300.9 | 911.4 |
| 16-20 | 67.8 | 323 | 614.4 | 35.5 | 204.4 | 523.4 |
| 20-24 | 26.9 | 175.6 | 530.5 | 15.8 | 119.8 | 298 |

TABLE 19

| | EXAMPLE 21 | | | Demadex ® 100 mg tablets | | |
|---|---|---|---|---|---|---|
| | Excretion Rate (μg/hr) | | | Excretion Rate (μg/hr) | | |
| Time | Min- | | | Min- | | |
| (hrs.) | imum | Mean | Maximum | imum | Mean | Maximum |
| 0-4 | 54.6 | 373.6 | 1180 | 908.1 | 1899.8 | 2849.9 |
| 4-8 | 76.7 | 1160 | 2113.8 | 166.1 | 784.4 | 1847.5 |
| 8-12 | 55.6 | 424.3 | 891.4 | 59.3 | 165.1 | 305.7 |
| 12-16 | 63.1 | 122.6 | 265.3 | 15.3 | 76.1 | 116.4 |
| 16-20 | 34.4 | 133.5 | 232.4 | 8.7 | 67.9 | 107 |
| 20-24 | 36.4 | 64.8 | 93 | 12.8 | 61.4 | 166.3 |

Table 20 lists the relative bioavailability in the Fasted and Fed states for Examples 20, 21, and 22 in comparison to Demadex®.

TABLE 20

| Relative | | Relative | |
|---|---|---|---|
| Bioavailability | | Bioavailability | |
| Fasted | (AUC) | Fed | (AUC) |
| Ratio | (%) | Ratio | (%) |
| Ex. 22/Demadex ® | 48.34 | Ex. 22/Demadex ® | 52.74 |
| Ex. 20/Demadex ® | 49.12 | Ex. 20/Demadex ® | 61.56 |
| Ex. 21/Demadex ® | 51.86 | Ex. 21/Demadex ® | 52.78 |

Table 21 lists the food effect differences for Examples 20, 21, 22 and the Demadex® formulation.

TABLE 21

| Food Effect | | | Food Effect | | |
|---|---|---|---|---|---|
| Difference | (Cmax) | (AUC) | Difference | (Cmax) | (AUC) |
| Ratio | (%) | (%) | Ratio | (%) | (%) |
| Ex. 22 Fasted/Ex. 22 | 36.53 | 81.49 | Ex. 22 Fed/Ex. 22 | 273.72 | 122.71 |
| Fed | | | Fasted | | |
| Ex. 20 Fasted/Ex. 20 | 89.99 | 70.94 | Ex. 20 | 111.12 | 140.96 |
| Fed | | | Fed/Ex. 20 Fasted | | |
| Ex. 21 Fasted/Ex. 21 | 52.44 | 87.36 | Ex. 21 Fed/Ex. 21 | 190.70 | 114.47 |
| Fed | | | Fasted | | |
| Demadex ® Fasted/ | 105.12 | 88.91 | Demadex ® Fed/ | 95.13 | 112.47 |
| Demadex ® Fed | | | Demadex ® Fasted | | |

Many other variations of the present invention will be apparent to those skilled in the art and are meant to be within the scope of the claims appended hereto.

1. A sustained release oral dosage form comprising:
an effective amount of torsemide or a pharmaceutically acceptable salt thereof and a sustained release excipient, said sustained release dosage form providing an in-vitro dissolution rate when measured by USP 26 (2003) dissolution Apparatus type III, in pH change media with an agitation of 15 dpm in 250 ml and at 37° C. which is from 0 to about 50% torsemide released after 1 hour; from about 1 to about 60% torsemide released after 3 hours; from about 5 to about 70% torsemide released after 7 hours; from about 10 to about 95% torsemide released after 12 hours; not less than about 25% torsemide released after 16 hours; and not less than about 35% torsemide released after 24 hours.

2. The sustained release oral dosage form of claim 1, wherein said sustained release excipient comprises a sustained release material selected from the group consisting of a gelling agent, a cellulose ether, an acrylic resin, a protein derived material, a wax, shellac, a sustained release polymer, an oil, and mixtures thereof.

3. The sustained release oral dosage form of claim 1; wherein said sustained release excipient comprises a gelling agent comprising at least one natural or synthetic gum.

4. The sustained release oral dosage form of claim 3, wherein said at least one natural or synthetic gum is selected from the group consisting of a heteropolysaccharide gum, a homopolysaccharide gum or a combination thereof.

5. The sustained release oral dosage form of claim 3, wherein said at least one natural or synthetic gum is a mixture of a heteropolysaccharide gum and a homopolysaccharide gum capable of cross-linking said heteropolysaccharide gum when exposed to said environmental fluid.

6. The sustained release oral solid dosage form of claim 3, further comprising an inert pharmaceutical diluent.

7. The sustained release oral solid dosage form of claim 6, wherein the ratio of said inert diluent to said gelling agent is from about 1:3 to about 3:1.

8. The sustained release oral solid dosage form of claim 3, further comprising an ionizable gel strength enhancing agent capable of crosslinking with said gelling agent and increasing the gel strength when the dosage form is exposed to said environmental fluid.

9. The sustained release oral solid dosage form of claim 5, wherein said heteropolysaccharide gum comprises xanthan gum and said homopolysaccharide gum comprises locust bean gum.

10. The sustained release oral dosage form of claim 1, further comprising an immediate release component which also comprises said torsemide or pharmaceutically acceptable salt thereof.

11. The sustained release oral dosage form of claim 10, wherein said sustained release oral dosage form is a bilayer tablet comprising a sustained release layer comprising torsemide or a pharmaceutically acceptable salt thereof and a sustained release excipient and an immediate release layer comprising said torsemide and a pharmaceutically acceptable diluent.

12. The sustained release oral dosage form of claim 1, wherein the in-vitro dissolution rate of the dosage form when measured by USP 26 (2003) dissolution Apparatus type III, in pH change media with an agitation of 15 dpm in 250 ml and at 37° C. which is from about 5 to about 44% torsemide released after 1 hour; from about 6 to about 46% torsemide released after 3 hours; from about 11 to about 54% torsemide released after 7 hours; from about 41 to about 91% torsemide released after 12 hours; not less than about 64% torsemide released after 16 hours; and not less than about 90% torsemide released after 24 hours.

13. The sustained release oral dosage form of claim 1, which provides a mean urinary excretion rate of torsemide of about 210 μg/hr to about 848 μg/hr from 0 to about 4 hours; about 290 μg/hr to about 1160 μg/hr from about 4 to about 8 hours; about 161 μg/hr to about 778 μg/hr from about 8 to about 12 hours; about 122 μg/hr to about 301 μg/hr from about 12 to about 16 hours; about 133 μg/hr to about 323 μg/hr from about 16 to about 20 hours; and about 64 μg/hr to about 182 μg/hr from about 20 to about 24 hours after single dose oral administration of the sustained release oral dosage form to human subjects.

14. A sustained release oral dosage form comprising:
an effective amount of torsemide or a pharmaceutically acceptable salt thereof and a sustained release excipient, said sustained release oral dosage form providing for the sustained release of said torsemide or pharmaceutically acceptable salt thereof for about 8 to about 24 hours and said dosage form providing a mean urinary excretion rate of torsemide of at least about 200 μg/hr for about 4 to about 20 hours after single dose oral administration to human subjects.

15. The sustained release oral dosage form of claim 14, wherein said dosage form provides a mean urinary excretion rate of torsemide of at least about 700 μg/hr for about 8 to about 12 hours after single dose oral administration of the sustained release oral dosage form to human subjects.

16. The sustained release oral dosage form of claim 14, wherein said dosage form provides a mean Cmax of torsemide of from about 1 μg/ml to about 7 μg/ml per 100 mg of torsemide upon single dose oral administration to human subjects.

17. The sustained release oral dosage form of claim 14, wherein said sustained release oral dosage form provides a mean Tₘₐₓ of torsemide at from about 1 to about 8 hours after single dose oral administration to human subjects.

18. The sustained release oral dosage form of claim 14, wherein said sustained release oral dosage form provides a mean AUC₍₀₋₂₄₎ of from about 10 μg.h/ml to about 40 μg.h/ml per 100 mg torsemide upon single dose oral administration to human subjects.

19. A method of treating congestive heart failure comprising administering the sustained release oral dosage form of claim 14 to a human patient suffering from congestive heart failure.

20. A method of treating edema comprising administering the sustained release oral dosage form of claim 14 to a human patient suffering from edema.

21. A bi-layer sustained release oral dosage form comprising:
a first layer comprising an effective amount of torsemide or a pharmaceutically acceptable salt thereof and a sustained release excipient;
a second layer comprising an effective amount of torsemide and an immediate release excipient,
said dosage form providing for the sustained release of said torsemide or pharmaceutically acceptable salt thereof for about 8 to about 24 hours when the dosage form is exposed to an environmental fluid and said dosage form providing a mean urinary excretion rate of torsemide of at least about 200 μg/hr for about 4 to about 20 hours after single dosage oral administration to human subjects.

22. The sustained release oral dosage form of claim 21, wherein said dosage form provides a mean Cmax of torsemide of from about 1 μg/ml to about 7 μg/ml per 100 mg of torsemide upon single dose oral administration to human subjects.

23. The sustained release oral dosage form of claim 21, wherein said sustained release oral dosage form provides a mean Tₘₐₓ of torsemide at from about 1 to about 8 hours after single dose oral administration to human subjects.

24. The sustained release oral dosage form of claim 21, wherein said sustained release oral dosage form provides a mean AUC₍₀₋₂₄₎ of from about 10 μg.h/ml to about 40 μg.h/ml per 100 mg torsemide upon single dose oral administration to human subjects.
